(19) United States
(12) Patent Application Publication
(10) Pub. No.: US 2023/0414384 A1
(43) Pub. Date: Dec. 28, 2023

(54) SEGMENTED BALLOON-EXPANDABLE STENT SYSTEM FOR PRESERVATION OF THE ARTERIAL LUMEN DURING BENDING

(71) Applicant: EFEMORAL MEDICAL, INC., LOS ALTOS, CA (US)

(72) Inventors: Lewis B. SCHWARTZ, Lake Forest, IL (US); Ivan TZVETANOV, Lake Forest, CA (US); Alex ESTRADA, Menlo Park, CA (US)

(21) Appl. No.: 18/037,313

(22) PCT Filed: Nov. 23, 2022
(86) PCT No.: PCT/US2021/060578

§ 371 Date: May 16, 2023

Related U.S. Application Data
(60) Provisional application No. 63/116,919, filed on Nov. 23, 2020.

(51) Int. Cl. A61F 2/852; A61F 2/958

(52) U.S. Cl. CPC A61F 2/852; A61F 2/958; A61F 2002/826

(57) ABSTRACT

Devices, systems, and methods are provided to maintain or enhance blood flow through the blood vessel. Balloon-expandable, bioresorbable, vascular stent elements that provides high radial force at the arterial wall while still preserving patency of the lumen during bending are described herein. Multiple, short, balloon-expandable scaffolds mounted in series on a delivery system and deployed simultaneously via a single balloon inflation are described. The individual scaffolds maintain the arterial lumen with high radial force while the inter-scaffold spaces are free to bend and compress during limb movement. The result is an artery in which the lumen is both adequately preserved and effectively stented.

### CROSS REFERENCES TO RELATED APPLICATION

This application claims the benefit and priority of U.S. Provisional Patent Application No. 63/116,919, entitled “SEGMENTED BALLOON-EXPANDABLE STENT SYSTEM FOR PRESERVATION OF THE ARTERIAL LUMEN DURING BENDING”, filed on Nov. 23, 2020, the full disclosure of the above referenced application is incorporated herein by reference.

### FIELD OF THE INVENTION

The present application pertains generally to the field of medical devices. More specifically, the present application pertains to the design and manufacture of intravascular stents intended to maintain patency (blood flow) of blood vessels (arteries and veins).

### BACKGROUND

Percutaneous peripheral intervention (PPI) has become the treatment-of-choice for symptomatic peripheral arterial occlusive disease (PAOD). This minimally invasive therapy provides equivalent pain relief and limb salvage as compared to surgical bypass grafting while minimizing patient morbidity, complications and cost. Unfortunately, its durability remains poor; after only one-year, approximately 50% of all PPI procedures will be attended by symptomatic recurrence and/or restenosis necessitating reintervention. In one recent study of the percutaneous treatment of long femoropopliteal occlusive lesions (>150 mm) with balloon angioplasty, only 34% remained patent and free from restenosis after only one year.

Most devices currently utilized in peripheral vascular intervention have been adaptations of devices originally designed for the coronary arteries. This strategy is problematic, however, as the peripheral arteries are larger, longer, more diffusely diseased and calcific, and bend and twist in more pronounced and less predictable ways than epicardial coronary arteries. It is, perhaps, not surprising that identifying the optimal device design for the effective treatment of the peripheral vasculature has been elusive.

In its current state, endovascular treatment of peripheral arteries is accomplished through a combination of plain balloon angioplasty, percutaneous atherectomy, paclitaxel-coated balloon angioplasty, plain nitinol stents, paclitaxel-coated nitinol stents and/or paclitaxel-eluting nitinol stents. Plain balloon angioplasty, while popular for the treatment of short lesions, is woefully ineffective in long lesions; one study documented a dismal 28% one-year patency in lesions with mean length of only 8.7 cm. Plain nitinol stents, originally designed to treat dissections and occlusions in “bail out” scenarios, don't fare much better. The continuous movement and outward force exerted by these ever-expanding permanent devices generates chronic inflammation, foreign body reaction, smooth muscle cell proliferation, restenosis and therapeutic failure. Indeed, a recent review concluded that peripheral arterial patency following self-expanding stent implantation is no better than plain balloon angioplasty alone.

Angioplasty balloons sprayed or dipped in pharmacologic compounds intended to attenuate neointimal hyperplasia have also been widely applied; to date, three separate, randomized, controlled clinical trials have demonstrated marginally enhanced patency compared to uncoated balloons. However, so-called “drug-coated balloons” rarely maintain patency in the long lesions routinely encountered in clinical practice. For example, one longitudinal study of drug-coated balloons inflated in lesions averaging 24 cm documented a requirement for unplanned stent implantation in 23% of cases and a dismal two-year primary patency of only 54%. Furthermore, a recent meta-analysis of paclitaxel-coated device trials has revealed that these drug-device formulations carry an increased risk of late mortality, possibly derived from long-term, low-level exposure to the cytotoxic drug. The analysis suggested that patients treated with paclitaxel-coated devices sustain significantly higher all-cause mortality than patients treated with bare devices when examined at two years (7.2% v. 3.8%) and at five years (14.7% v. 8.1%). Once confirmed, this finding led a United States Food & Drug Administration Consensus Panel to conclude that, “a late mortality signal associated with the use of paclitaxel-coated devices to treat femoropopliteal PAOD was present.” This finding has led to a significant dampening of enthusiasm for their use leaving patients and interventionists with far fewer treatment options for this debilitating disease.

Efforts at coating self-expanding metal stents with paclitaxel have likewise proven disappointing. A simple strategy in which the drug is sprayed on a slotted tube nitinol stent without an excipient, was tested in a randomized, pivotal trial using bare metal stents as comparators. The initial results were favorable; in a cohort of 6.5 cm lesions, the device exhibited superior one-year patency to its bare nitinol counterpart (89.9% v. 73.0%; p<0.01). Unfortunately, given the short elution profile of the drug and the persistent metal foreign body, the results quickly deteriorated and, by three-years, the patency rate was only 75%. Moreover, the real-world, global experience with the device has been consistently underwhelming. In one international study of 690 patients with a mean lesion length of 17 cm, the one- and three-year restenosis rates were 36% and 51%, respectively, leading the authors to conclude that the device performed no better than bare nitinol.

The final and most recent device to achieve market approval in the U.S. is a paclitaxel-eluting device. The nitinol device is coated with 0.167 μg paclitaxel/mm 2 stent surface area imbedded in the PVDF-HFP fluoropolymer utilized in successful coronary applications. Although the initial clinical results in Europe were promising, the recently-reported results of trial weren't as favorable. After treatment of patients with lesions averaging 8.6 cm, the primary one-year patency was only 88% and the small, nominal difference in reintervention rate between the device and its control arm was not statistically significant.

All of these intravascular stents share the same basic metal platform: slotted-tube nitinol. Its persistent popularity in the peripheral vasculature is based on the long-held belief that more rigid, balloon-expandable stents cannot be safety implanted in arteries that bend. Given the well-documented twisting forces generated in peripheral arteries, it has been believed that any attempt at implantation of rigid, balloon-expandable stents would result in plastic deformation, restenosis, thrombosis and/or pseudoaneurysm formation. A recent report endorsing the “standards of practice for superficial femoral and popliteal artery angioplasty and stenting,” suggested that, “balloon-expandable metal stents are no longer used in the femoropopliteal segment because of the risk of external compression and longitudinal axis deformation.”

Therefore, it would be advantageous to have a balloon-expandable stent that can be safely used in highly mobile vasculature. At least some of these objectives will be met by the embodiments described below.

### SUMMARY

The embodiments herein describe a device for placement within a blood vessel to maintain or enhance blood flow through the blood vessel. The device may comprise one or more balloon-expandable, bioresorbable, vascular stent elements configured to be implanted in the blood vessel as a stent. The stent may be configured to provide high radial force at the blood vessel wall while still preserving patency of the lumen during bending. In an embodiment, bending of the blood vessel is accommodated by bending of spaces between the stent elements. In an embodiment, axial compression of the blood vessel is absorbed by axial compression of both the stent elements and spaces between the stent elements. The stent elements may be comprised of a bioresorbable material. Alternatively, the stent elements may be comprised of a permanent material.

This and other aspects of the present disclosure are described herein.

### BRIEF DESCRIPTION OF THE DRAWINGS

Present embodiments have other advantages and features which will be more readily apparent from the following detailed description and the appended claims, when taken in conjunction with the accompanying drawings, in which:

FIG. 1 shows the typical radial resistive forces of intravascular stents.

FIG. 2A illustrates one embodiment of a multi-element stent. FIG. 2B is a magnified view of the stent elements in FIG. 2A.

FIGS. 3A-3C depict deployment of a balloon-expandable multi-element stent.

FIG. 4A shows an implanted multi-element stent in a popliteal artery during full flexion of the hip and knee. FIG. 4B depicts the implanted device of FIG. 4A shown in three dimensions.

FIGS. 5A-5D show an embodiment of a stent pattern. FIG. 5A is a two-dimensional depiction of an element. FIG. 5B shows a magnified view of the cells in FIG. 5A. FIGS. 5C and 5D show the stent element of FIG. 5A in cylindrical form.

FIG. 6 shows a laser cut stent.

FIGS. 7A-7C show an angiographic example of a segmented, balloon-expandable, intravascular stent.

FIG. 8 shows axial compression (shortening) of inter-scaffold spaces during porcine hind limb flexion.

FIG. 9 shows axial compression (shortening) of scaffolds during porcine hind limb flexion.

FIGS. 10A and 10B show a five-segment device created by crimping five individual scaffolds onto a single delivery system

FIGS. 10C and 10D show a segmented, balloon-expandable, intravascular stent system that provides high radial force at the arterial wall while still preserving patency of the lumen during bending (five serial scaffolds).

FIGS. 10E and 10F show a control, self-expanding nitinol stent implanted into the iliofemoral artery of a swine.

FIG. 10G shows bend angle measurement after deployment of self-expanding stents (Nitinol) and 5-scaffold balloon-expandable devices in a porcine model of percutaneous peripheral vascular intervention.

FIG. 10H shows target artery axial compression after deployment of self-expanding stents (Nitinol) and 5-scaffold balloon-expandable devices in a porcine model of percutaneous peripheral vascular intervention.

FIG. 10I shows minimum and mean target artery lumen diameter after deployment of self-expanding stents (Nitinol) and 5-scaffold balloon-expandable devices in a porcine model of percutaneous peripheral vascular intervention.

FIGS. 10J and 10K show angiographic images of bilateral porcine femoral arteries treated with 5-scaffold balloon-expandable devices and self-expanding stents (Nitinol) after 90-days.

FIG. 10L shows angiographic maximum diameter stenosis after implantation of the 5-scaffold device vs. nitinol SES in the porcine iliofemoral model.

FIG. 10M shows serial optical coherence tomography (OCT) to image scaffold degradation over time.

FIGS. 10N and 10O show photomicrographs of porcine femoral arteries treated with the 5-scaffold device (FIG. 10N) or nitinol stents (FIG. 10O) and harvested after 2 years.

FIG. 11 is a schematic diagram of a micro-stereolithograph used to create a stent, according to one embodiment.

### DETAILED DESCRIPTION

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

The word “exemplary” is used herein to mean “serving as an example, instance, or illustration.” Any implementation described herein as “exemplary” is not necessarily to be construed as advantageous over other implementations.

Various embodiments are described herein with reference to the figures. The figures are not drawn to scale and are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

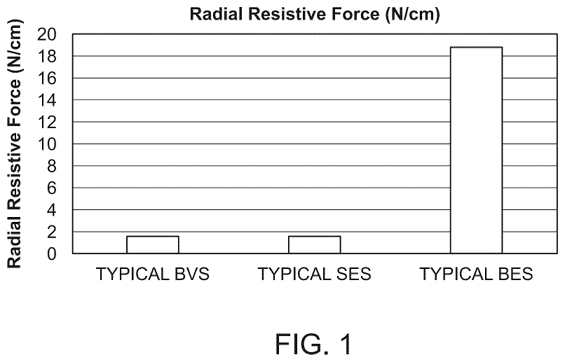

FIG. 1 shows the typical radial resistive forces of intravascular stents. A typical “bioresorbable vascular scaffold” (BVS) or absorbable stent has a radial resistive force of under 2 N/cm. Similarly, a typical self-expanding metal stent (SES) has a radial resistive force of under 2 N/cm. Typical balloon-expandable metal stents (BES) have a much higher radial resistive force, sometimes above 18 N/cm.

The long, peripheral arteries of mammals bend, compress and twist in order to preserve blood flow during limb movement. Intravascular devices intended to reside within these arteries must, therefore, be flexible enough to accommodate repeated bending and deformation. However, flexible intravascular devices do not typically provide the radial strength necessary to reliably maintain the flow channels of severely diseased arteries.

Described herein is the design of a segmented, balloon-expandable, intravascular stent system that provides high radial force at the arterial wall while still maintaining patency of the lumen during bending. This is afforded using multiple, short, balloon-expandable scaffolds mounted in series on a delivery system and deployed simultaneously via a single balloon inflation. The individual scaffolds preserve the arterial lumen with high radial force while the inter-scaffold spaces absorb the bending and compression that accompanies limb movement.

The embodiments herein describe the design of a segmented, balloon-expandable, intravascular stent system that provides high radial force at the arterial wall while still preserving patency of the lumen during bending. A critical design element of the individual scaffold segments is the provision of radial strength more typical of highly effective, rigid, balloon-expandable stents as opposed to weaker self-expanding stents.

In contrast to most stent patterns which are designed to marry both radial force and longitudinal flexibility, the patterns described herein are specifically tailored to maximize radial force and rigidity and forego longitudinal and axial flexibility.

The devices described herein are multi-element, vascular stents (or “vascular scaffolds”). These stents are comprised of multiple, short, rigid, cylindrical stent segments, or elements, which are separate from one another but may be referred to together as a multi-element stent.

Generally, at least two of the elements of the multi-element stent described herein will be sufficiently rigid to provide a desired level of strength to withstand the stresses of the vessel in which they are placed, such as a tortuous peripheral vessel. At the same time, a multi element stent will also be flexible, due to the fact that it is made up of multiple separate elements, thus allowing for placement within a curved, torturous blood vessel. In some embodiments, at least two of the elements vary in rigidity or radial strength in a multi-element stent. In one embodiment, the outer elements may have a lesser radial strength than the inner elements in a multi-element stent. In another embodiment, a multi-element stent comprises elements having an increasing radial strength serially along the length of the multi-element stent, such as in an AV fistula. Thus, the radial strength of elements may vary and be tailored by known characteristics of a target artery.

Additionally, the multi element stents described herein will usually be balloon-expandable rather than self-expanding, since balloon-expandable stents are typically stronger than self-expanding stents. Each balloon expandable element of the stent may have relatively high radial force (rigidity) due to the described structures and materials. A stent element is defined as being radially rigid if it has a radial strength significantly higher than self-expanding stents that is similar or greater in magnitude to that of traditional, metal balloon-expandable stents, such as those made of steel or cobalt-chromium.

When mounted serially on an inflatable balloon, they can be simultaneously implanted side-by-side in long blood vessels. During motion of the organism, the elements can move independently, maintaining their individual shape and strength while the intervening, non-stented elements of the vessel can twist, bend and rotate unencumbered. The result is a treated vessel with a rigidly maintained flow channel that still enjoys unrestricted flexibility during organismal movement.

The described embodiments exploit the principles that, (1) a rigid device that is deployed via balloon-expansion represents the optimal design of an intravascular stent given its transient effect on the arterial wall and relative ease of precise implantation, (2) a long, rigid device cannot be safely implanted in an artery that bends and twists with skeletal motion, (3) long arteries that bend and twist could be effectively treated with multiple, short BES that allow the intervening, non-stented arterial elements to move unencumbered, (4) the length, number and spacing of the stent elements could be determined by the known and predictable bending characteristics of the target arteries, and (5) arteries need only be scaffolded transiently; late dissolution of the stent will have little effect on the long-term effectiveness of treatment.

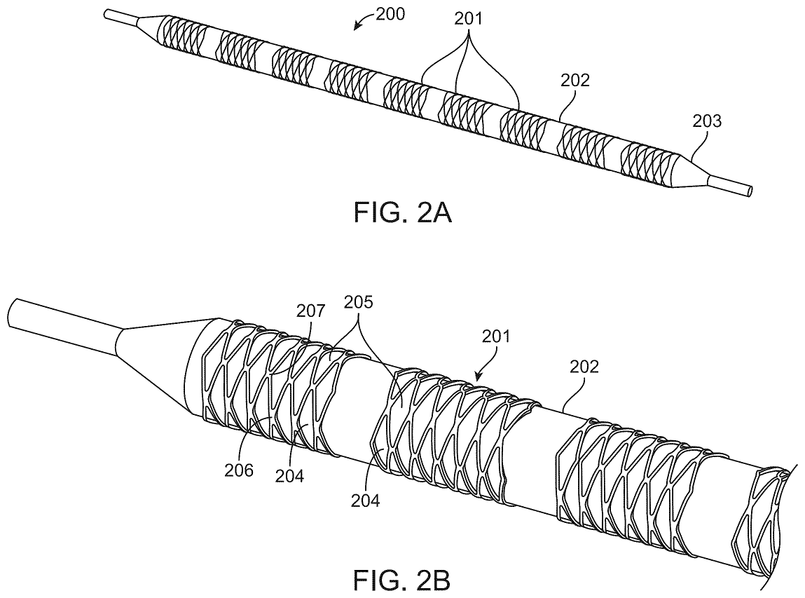

One embodiment of the fully assembled device in shown in FIG. 2A. A single balloon inflation and device deployment can treat a long segment of diseased artery while still preserving the critical ability of the artery to bend with skeletal motion such as sitting or walking. Multi-element stent 200 comprises multiple stent elements 201. Individual balloon-expandable stent elements 201 are crimped onto an inflatable balloon 203 to facilitate delivery. FIG. 2B is a magnified view of the stent elements 201 in FIG. 2A. Individual elements 201 are positioned serially along a longitudinal length of the balloon 203 and spaced such that the stent elements 201 do not touch one another. Further, the spacing is such that after deployment, the stent elements 201 do not touch or overlap during skeletal movement. The number of elements 201, length of elements, and gap 202 between elements 201 may vary depending on the target vessel location. In an embodiment, each element 201 in the multi-element stent 200 has the same length. In multi-element stents having three or more elements 201, and thus two or more gaps 202, the gaps may be of the same length.

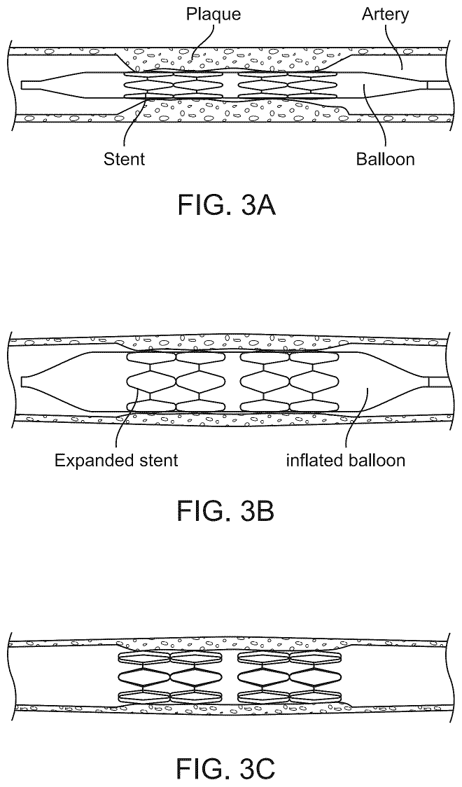

FIGS. 3A-3C depict deployment of a balloon-expandable multi-element stent. In FIG. 3A a multi-element stent mounted on a balloon is advanced to the lesion. In FIG. 3B the balloon and stent are expanded. In FIG. 3C the balloon is withdrawn leaving the multi-element stent still within the artery.

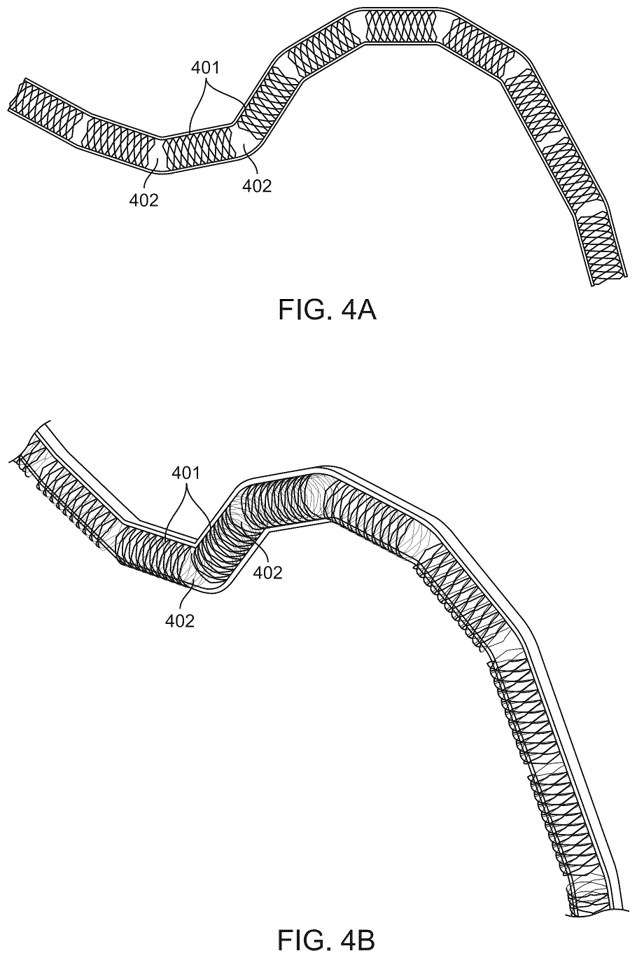

FIG. 4A shows an implanted multi-element stent in a popliteal artery during full flexion of the hip and knee. FIG. 4B depicts the implanted device of FIG. 4A shown in three dimensions. The individual stent elements 401 are spaced such that they do not overlap even when the artery is highly bent. Unencumbered arterial movement is afforded through flexion or extension of the unstented gaps 402.

Stent elements may comprise various shapes and configurations. Some or all of the stent elements may comprise closed-cell structures formed by intersecting struts. Closed-cell structures may comprise diamond, square, rectangular, parallelogrammatic, triangular, pentagonal, hexagonal, heptagonal, octagonal, clover, lobular, circular, elliptical, and/or ovoid geometries. Closed-cells may also comprise slotted shapes such as H-shaped slots, I-shaped slots, J-shaped slots, and the like. Additionally or alternatively, stent may comprise open cell structures such as spiral structures, serpentine structures, zigzags structures, etc. Strut intersections may form pointed, perpendicular, rounded, bullnosed, flat, beveled, and/or chamfered cell corners. In an embodiment, stent may comprise multiple different cells having different cell shapes, orientations, and/or sizes. Various cell structures have been described in PCT International Application Number PCT/US16/20743, entitled “MULTI-ELEMENT BIORESORBABLE INTRAVASCULAR STENT”, PCT International Application Number PCT/US20/19132, entitled “ABSORBABLE INTRAVASCULAR DEVICES THAT EXHIBIT THEIR GREATEST RADIAL STRENGTH AT THEIR NOMINAL DIAMETERS”, and PCT International Application Number PCT/US19/35861, entitled “ABSORBABLE INTRAVASCULAR DEVICES THAT SHORTEN UPON EXPANSION CREATING SPACE FOR VASCULAR MOVEMENT”, the full disclosures of which are herein incorporated by reference.

Returning to FIG. 2B, in this exemplary embodiment, the stent elements 201 have a diamond shaped closed-cell pattern. Elements 201 comprise intermixed diamond shaped closed cells 204, 205. Diamond shaped cells 204 may be aligned in the longitudinal and/or the circumferential directions in a repeating pattern. Similarly, diamond shaped cells 205 may be aligned in the longitudinal and/or the circumferential directions in a repeating pattern. Additionally or alternatively, diamond shaped cells 204 and diamond shaped cells 205 may be helically aligned in an alternating pattern. In an embodiment, diamond shaped cells 204 and diamond shaped cells 205 are circumferentially offset. Additionally, diamond shaped cells 205 may be formed at a central location between four adjacent diamond shaped cells 204. The width of struts 206 between two corners of longitudinally aligned diamond shaped cells 204 are larger than the width of struts 207 between two corners of longitudinally aligned diamond shaped cells 205.

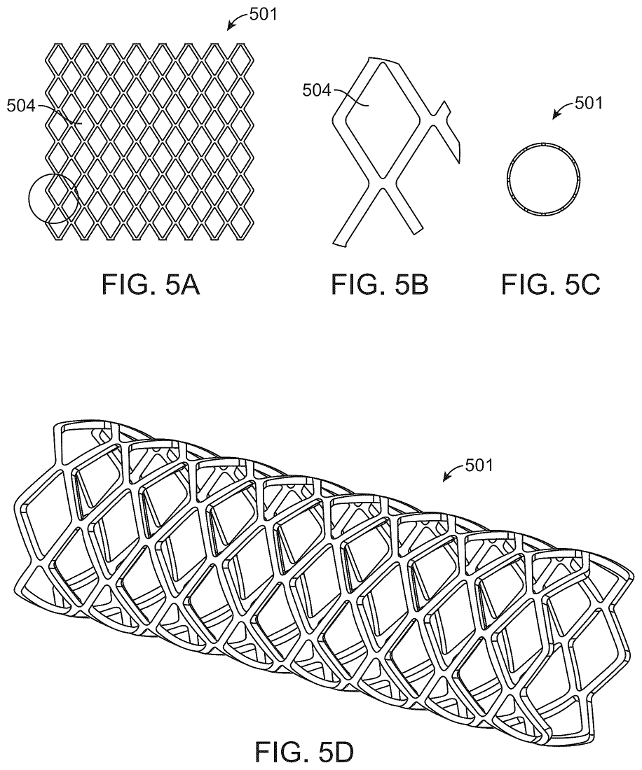

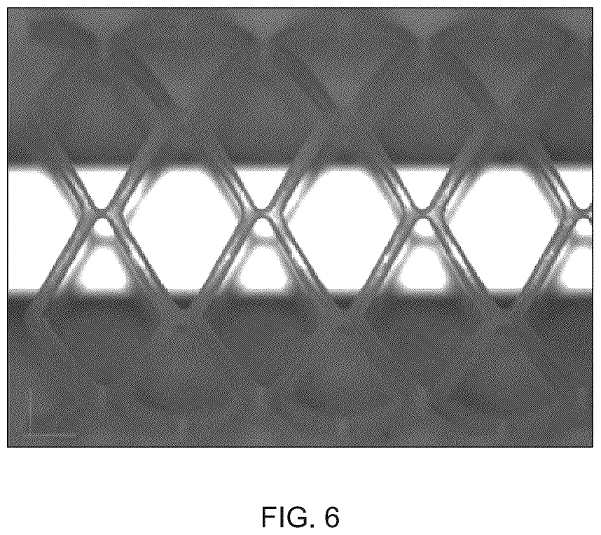

One embodiment of a stent pattern is shown in shown in FIGS. 5A-5D. The stent elements 501 have a diamond shaped closed-cell pattern with relatively thick strut widths and obliquely-angled links. Elements 501 comprise diamond shaped closed cells 504. Elements 501 may comprise wide struts 506 of 225 microns or larger. Elements 501 may similarly comprise thick struts 506 of 225 microns or larger. In an embodiment, elements 501 comprise struts 506 with a width and/or thickness of approximately 250 microns. The width and/or the height of struts 506 between two corners of diamond shaped cells 504 may be larger or smaller than the width and/or height of struts 506 forming the sides of diamond shaped cells 504. When compressed radially (crimped), most of the struts 506 are oriented horizontally. When expanded, however, the struts 506 become oriented in the vertical direction and, like the columns of a building, lend additional resistance to compression. The compressive load is spread throughout the repeating structure making it highly resistant to deformation. The stent pattern is designed for maximal radial force and stiffness when dilated to its nominal diameter. An example of an actual laser-cut stent designed herein is shown in FIG. 6.

The stents described herein may be formed from various different materials. In an embodiment, stents may be formed a polymer or co-polymer. In various alternative embodiments, the stent or stent element may be made from any suitable bioresorbable material such that it will dissolve non-toxically in the human body, such as but not limited to polyesters such as Polylactic acid, Poly(ε-caprolactone), Polyglycolic acid, and Polyhydroxyalkanoate, amino acid based polymers such as Polyesteramide, polycarbonates such as Polytrimethylene carbonate as well as any and all copolymers of the types described herein. In alternative embodiments, the stents may be formed from a permanent material such as a metal.

In various embodiments, any suitable polymer or copolymer may be used to construct the stent. The term “polymer” is intended to include a product of a polymerization reaction inclusive of homopolymers, copolymers, terpolymers, etc., whether natural or synthetic, including random, alternating, block, graft, branched, cross-linked, blends, compositions of blends and variations thereof. The polymer may be in true solution, saturated, or suspended as particles or supersaturated in the beneficial agent. The polymer can be biocompatible, or biodegradable. For purpose of illustration and not limitation, the polymeric material may include, but is not limited to, L-lactide, poly(D-lactic acid) (PDLA), poly(D,L-lactic acid) (PDLLA), poly(iodinated desamino tyrosyl-tyrosine ethyl ester) carbonate, poly(lactic-co-glycolic acid) (PLGA), poly(iodinated desaminotyrosyl-tyrosine ethyl ester) carbonate, salicylate based polymer, semicrystalline polylactide, phosphorylcholine, ε-caprolactone, polycaprolactone (PCL), poly-D,L-lactic acid, poly-L-lactic acid, poly(lactideco-glycolide), poly(hydroxybutyrate), poly(hydroxybutyrate-co-valerate), polydioxanone (PDS), polyorthoester, polyanhydride, poly(glycolic acid), poly(glycolic acid-co-trimethylene carbonate), polyphosphoester, polyphosphoester urethane, poly(amino acids), cyanoacrylates, poly(trimethylene carbonate), poly(iminocarbonate), polyalkylene oxalates, polyphosphazenes, polyiminocarbonates, and aliphatic polycarbonates, fibrin, fibrinogen, cellulose, starch, collagen, polyurethane including polycarbonate urethanes, polyethylene, polyethylene terephthalate, ethylene vinyl acetate, ethylene vinyl alcohol, silicone including polysiloxanes and substituted polysiloxanes, polyethylene oxide, polybutylene terephthalate-co-PEG, PCL-co-PEG, PLA-co-PEG, PLLA-co-PCL, polyacrylates, polyvinyl pyrrolidone, polyacrylamide, and combinations thereof. Non-limiting examples of other suitable polymers include thermoplastic elastomers in general, polyolefin elastomers, EPDM rubbers and polyamide elastomers, and biostable plastic material including acrylic polymers, and its derivatives, nylon, polyesters and expoxies. In some embodiments, the stent may include one or more coatings, with materials like poly-L-lactide (PLLA) or poly(D,L-lactic acid) (PDLLA). These materials are merely examples, however, and should not be seen as limiting the scope of the invention. The coating may comprise a drug and a solvent capable of dissolving the drug and swelling or softening the scaffold structural polymer. The solvent may be any single solvent or a combination of solvents. For purpose of illustration and not limitation, examples of suitable solvents include water, aliphatic hydrocarbons, aromatic hydrocarbons, alcohols, ketones, dimethyl sulfoxide, tetrahydrofuran, dihydrofuran, dimethylacetamide, acetonitrile, acetates, and combinations thereof.

The therapeutic drug may be any agent intended to prevent or attenuate pathologic consequences of intraluminal intervention such as inflammation, cell dysfunction, cell activation, cell proliferation, neointimal formation, thickening, late atherosclerotic change and/or thrombosis. In an embodiment, the drug may be Sirolimus and/or its derivatives. Examples of such therapeutic agents include, but are not limited to, antithrombotics, anticoagulants, antiplatelet agents, anti-lipid agents, thrombolytics, antiproliferatives, anti-inflammatories, agents that inhibit hyperplasia, smooth muscle cell inhibitors, antibiotics, growth factor inhibitors, cell adhesion inhibitors, cell adhesion promoters, antimitotics, antifibrins, antioxidants, anti-neoplastics, agents that promote endothelial cell recovery, matrix metalloproteinase inhibitors, anti-metabolites, antiallergic substances, viral vectors, nucleic acids, monoclonal antibodies, inhibitors of tyrosine kinase, antisense compounds, oligonucleotides, cell permeation enhancers, hypoglycemic agents, hypolipidemic agents, proteins, nucleic acids, agents useful for erythropoiesis stimulation, angiogenesis agents, anti-ulcer/anti-reflux agents, and anti-nauseants/anti-emetics, PPAR alpha agonists such as fenofibrate, PPAR-gamma agonists selected such as rosiglitazaone and pioglitazone, sodium heparin, LMW heparins, heparoids, hirudin, argatroban, forskolin, vapriprost, prostacyclin and prostacylin analogues, dextran, D-phe-pro-arg-chloromethylketone (synthetic anti-thrombin), glycoprotein IIb/IIIa (platelet membrane receptor antagonist antibody), recombinant hirudin, thrombin inhibitors, indomethacin, phenyl salicylate, beta-estradiol, vinblastine, ABT-627 (astrasentan), testosterone, progesterone, paclitaxel, methotrexate, fotemusine, RPR-101511A, cyclosporine A, vincristine, carvediol, vindesine, dipyridamole, methotrexate, folic acid, thrombospondin mimetics, estradiol, dexamethasone, metrizamide, iopamidol, iohexol, iopromide, iobitridol, iomeprol, iopentol, ioversol, ioxilan, iodixanol, and iotrolan, antisense compounds, inhibitors of smooth muscle cell proliferation, lipid-lowering agents, radiopaque agents, antineoplastics, HMG CoA reductase inhibitors such as lovastatin, atorvastatin, simvastatin, pravastatin, cerivastatin and fluvastatin, and combinations thereof.

Examples of antithrombotics, anticoagulants, antiplatelet agents, and thrombolytics include, but are not limited to, sodium heparin, unfractionated heparin, low molecular weight heparins, such as dalteparin, enoxaparin, nadroparin, reviparin, ardoparin and certaparin, heparinoids, hirudin, argatroban, forskolin, vapriprost, prostacyclin and prostacylin analogues, dextran, D-phe-pro-arg-chloromethylketone (synthetic antithrombin), dipyridamole, glycoprotein IIb/IIIa (platelet membrane receptor antagonist antibody), recombinant hirudin, and thrombin inhibitors such as bivalirudin, thrombin inhibitors, and thrombolytic agents, such as urokinase, recombinant urokinase, pro-urokinase, tissue plasminogen activator, ateplase and tenecteplase.

Examples of cytostatic or antiproliferative agents include, but are not limited to, rapamycin and its analogs, including everolimus, zotarolimus, tacrolimus, novolimus, ridafrolimus, temsirolimus, and pimecrolimus, angiopeptin, angiotensin converting enzyme inhibitors, such as captopril, cilazapril or lisinopril, calcium channel blockers, such as nifedipine, amlodipine, cilnidipine, lercanidipine, benidipine, trifluperazine, diltiazem and verapamil, fibroblast growth factor antagonists, fish oil (omega 3-fatty acid), histamine antagonists, lovastatin, topoisomerase inhibitors, such as etoposide and topotecan, as well as antiestrogens such as tamoxifen.

Examples of anti-inflammatory agents include, but are not limited to, colchicine and glucocorticoids, such as betamethasone, cortisone, dexamethasone, budesonide, prednisolone, methylprednisolone and hydrocortisone. Non-steroidal anti-inflammatory agents include, but are not limited to, flurbiprofen, ibuprofen, ketoprofen, fenoprofen, naproxen, diclofenac, diflunisal, acetominophen, indomethacin, sulindac, etodolac, diclofenac, ketorolac, meclofenamic acid, piroxicam and phenylbutazone.

Examples of antineoplastic agents include, but are not limited to, alkylating agents including altretamine, bendamucine, carboplatin, carmustine, cisplatin, cyclophosphamide, fotemustine, ifosfamide, lomustine, nimustine, prednimustine, and treosulfin, antimitotics, including vincristine, vinblastine, paclitaxel, docetaxel, antimetabolites including methotrexate, mercaptopurine, pentostatin, trimetrexate, gemcitabine, azathioprine, and fluorouracil, antibiotics, such as doxorubicin hydrochloride and mitomycin, and agents that promote endothelial cell recovery such as estradiol.

Antiallergic agents include, but are not limited to, permirolast potassium nitroprusside, phosphodiesterase inhibitors, prostaglandin inhibitors, suramin, serotonin blockers, steroids, thioprotease inhibitors, triazolopyrimidine, and nitric oxide.

That the segmented, balloon-expandable device would preserve the arterial lumen during bending was demonstrated in the experimental animal. Peripheral contrast angiography was performed in four female domestic farm pigs weighing between 25 and 35 kg. After induction of general anesthesia, intubation and mechanical ventilation, the carotid artery was surgically exposed with the animal in dorsal recumbency. A sheath was inserted into the common carotid artery under direct vision and advanced to the aortic bifurcation using fluoroscopy. Heparin was administered to achieve an activated clotting time >300 s. Nitroglycerin boluses were administered to mitigate secondary arterial vasospasm. Anteroposterior angiographic images were obtained in the neutral position with the hind limb naturally extended and repeated after manual, exaggerated hip and knee flexion (crouch position). Scaffolds were deployed into optimally-sized regions of the bilateral iliofemoral arteries using balloon inflation necessary to achieve complete wall apposition. Following device deployment and balloon withdrawal, angiography was repeated with the hind limb in both extension and flexion. Retrospective quantitative vascular analysis was used to assess the deformations of arteries, scaffolds and inter-scaffold spaces. Measurements included diameters and lengths of scaffolds, the intervening spaces between scaffolds and the proximal and distal arterial margins. Axial compression was defined as the difference between arterial target segment lengths in the neutral, extended position minus the length in the flexed position divided by length in the neutral position. Bend angle was defined as the approximate angle between the proximal and distal border of the sample target arterial segment.

A total of 38 resorbable scaffolds were implanted into 8 iliofemoral arteries of 4 animals. Devices were implanted in a configuration of 2 serial scaffolds in 2 arteries, 4 scaffolds in 2 arteries, 6 scaffolds in 3 arteries and 8 scaffolds in 1 artery. Total scaffolded arterial length ranged from 32 mm to 97 mm.

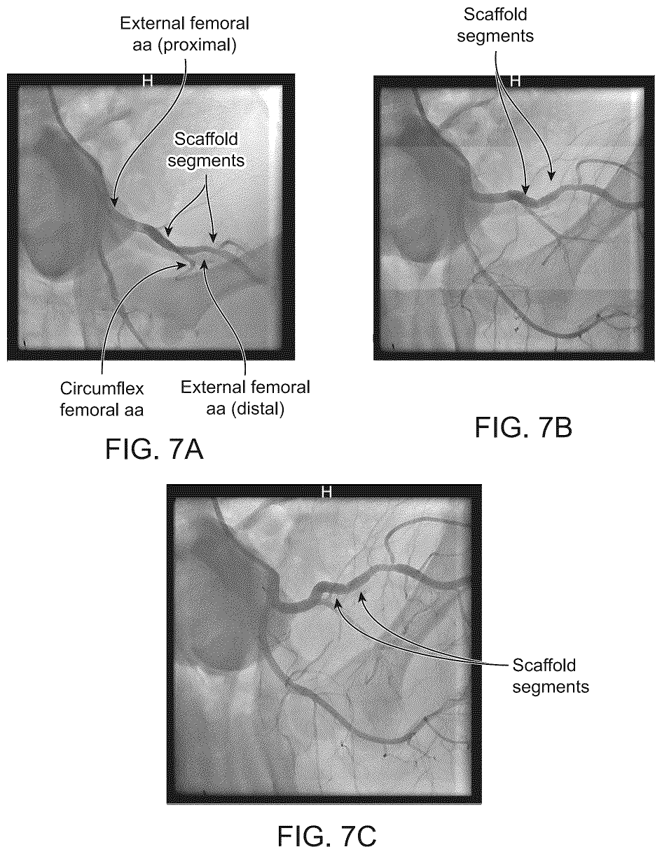

Following scaffold implantation, hind limb flexion produced predictable patterns of arterial deformation; an angiographic example is shown in FIGS. 7A, 7B, and 7C. A segmented, balloon-expandable, intravascular stent system is shown that provides high radial force at the arterial wall while still preserving patency of the lumen during bending (two serial scaffolds). Side-by-side scaffolds were percutaneously implanted into the left femoral artery of a farm swine. In the left panel, the left hind limb is extended. When the hind limb is manually flexed (right panel), the arterial bending is absorbed by axial shortening of the scaffolds and intervening space. Even when the hind limb is manually flexed to a non-physiologic position (bottom panel), the scaffolded segment remains widely patent.

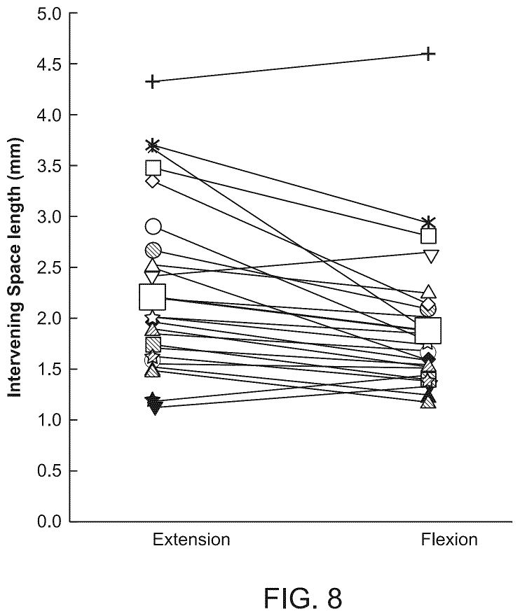

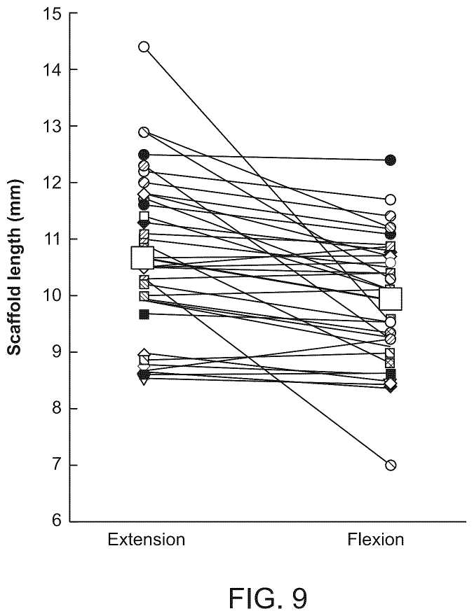

Despite containing multiple rigid scaffolds, the luminal arterial diameter of treated arteries remained preserved without kinking or occlusion even during extreme flexion (mean lumen diameter in extension 4.8±0.3 mm vs. mean lumen diameter in flexion 4.7±0.3). Individual length measurements of the scaffolds and inter-scaffold spaces were undertaken in order to assess which specific components of the system were mechanically absorbing the deformation. The results showed that the bending and axial compression of the artery was borne by shortening of the spaces between scaffolds (n=30 spaces, mean length in extension 2.2±08 mm vs. mean length in flexion 1.9±0.7 mm; p=0.0008 using paired t-test) as well as axial shortening of the scaffolds themselves (n=38 scaffolds; mean length in extension 10.7±1.4 mm vs. mean length in flexion 9.9±1.1 mm; p=0.0003 using paired t-test). The shortening of the individual components of the devices is depicted graphically in FIGS. 8 and 9.

FIG. 8 shows axial compression (shortening) of inter-scaffold spaces during porcine hind limb flexion. Axial compression (shortening) of the 30 intervening spaces between resorbable scaffolds during porcine hind limb flexion. Measurements were derived from angiographic images. The bold black squares connected by a solid black line represent the means.

FIG. 9 shows axial compression (shortening) of scaffolds during porcine hind limb flexion. Axial compression (shortening) of the 38 implanted resorbable scaffolds during porcine hind limb flexion. Measurements were derived from angiographic images. The bold black squares connected by a solid black line represent the means.

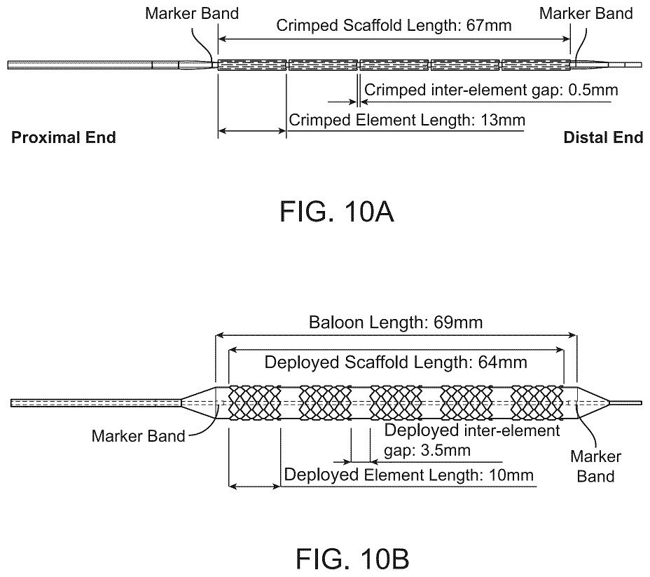

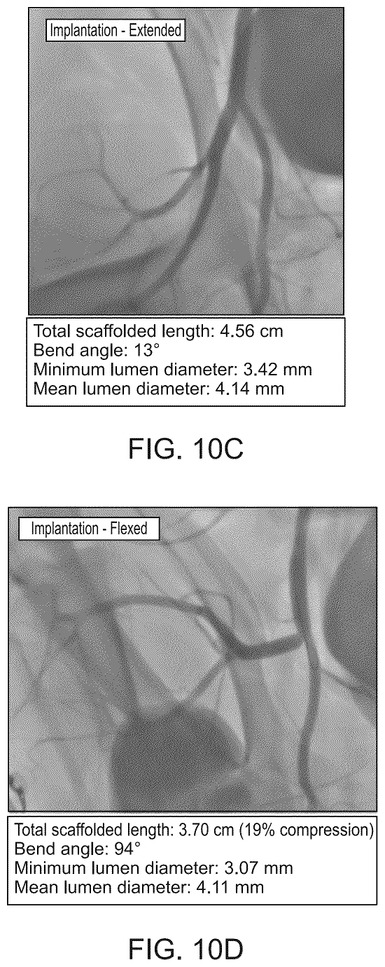

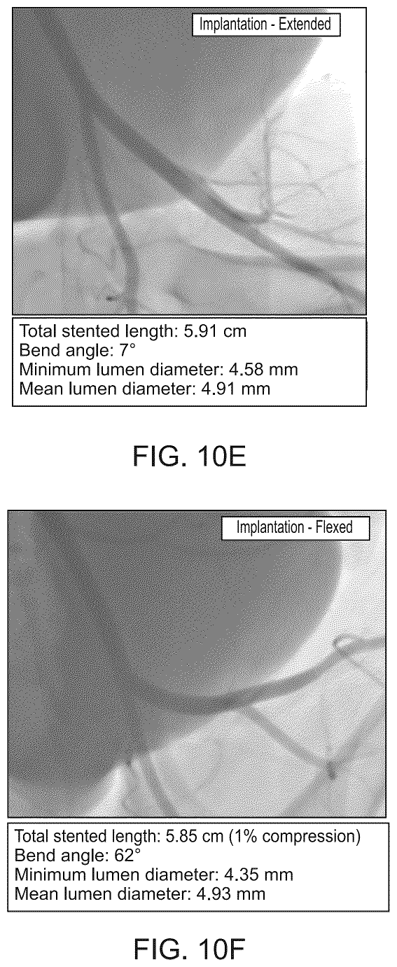

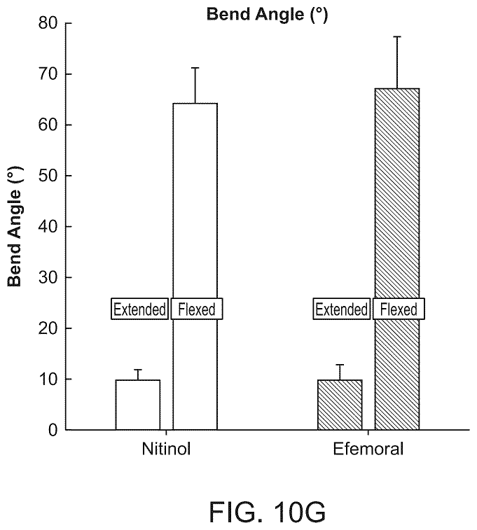

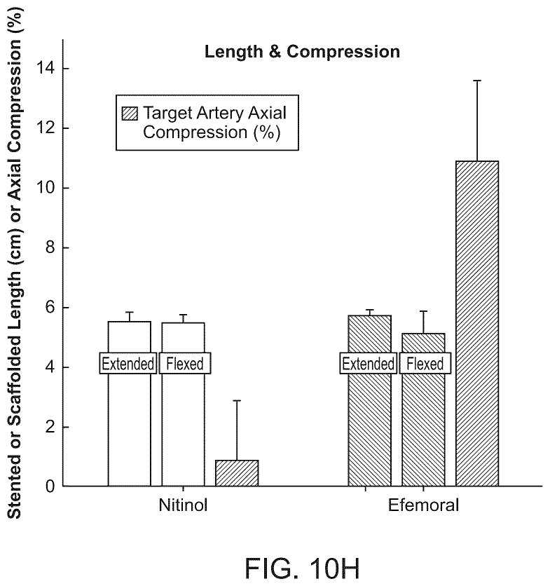

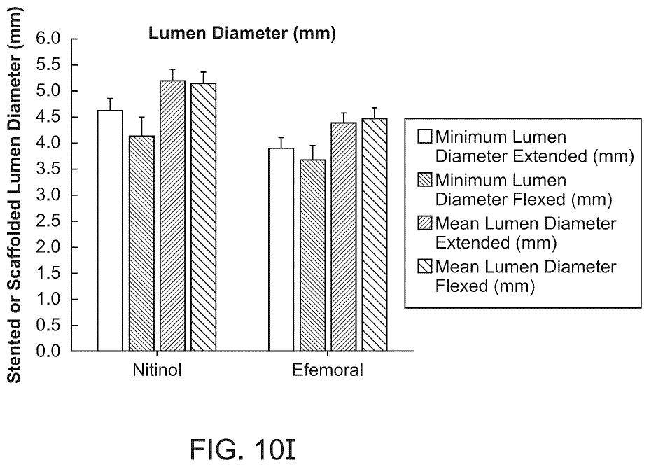

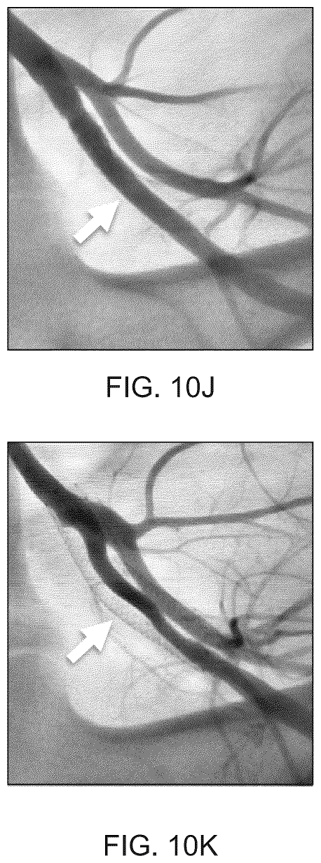

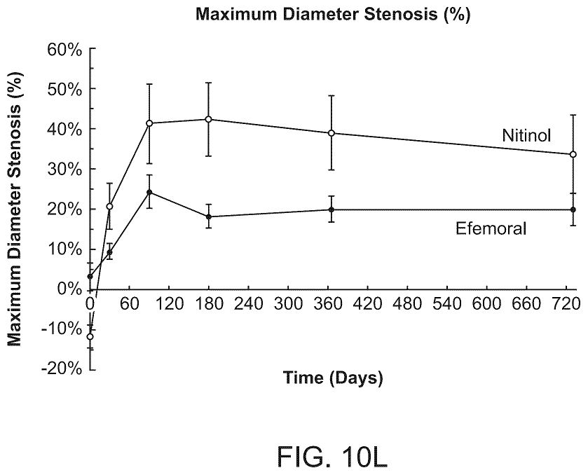

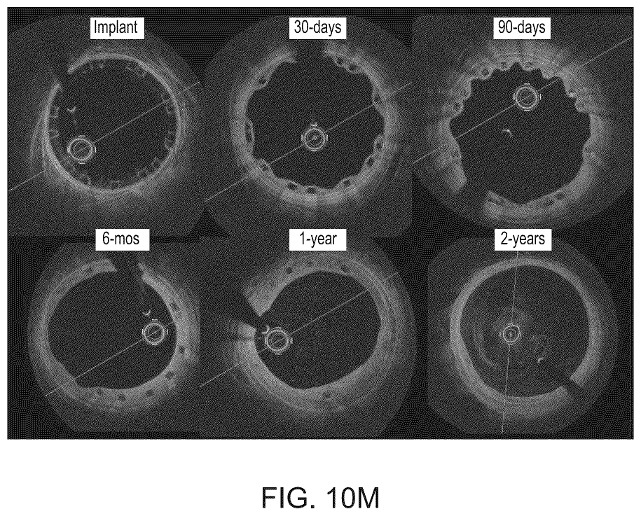

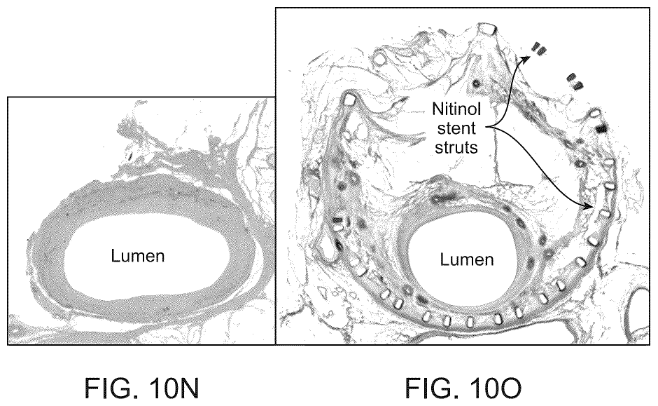

This same phenomenon was also demonstrated using a 5-scaffold device in a similar experimental model shown in FIGS. 10A and 10B. In this study, 8 female Yucatan mini-swine were anesthetized as above and 5-scaffold paclitaxel-eluting device implanted endovascularly into iliofemoral arteries via open carotid cut down FIGS. 10C and 10D.

Five scaffolds were simultaneous deployed in a target porcine iliofemoral artery via a single balloon inflation. The artery remained widely patent when the hind limb was extended (FIG. 10C) as well as maximally flexed (FIG. 10D). Numerical data from quantitative vascular angiographic analysis (QVA) is shown in each figure.

To serve as control, standard, approved, properly-sized, 6 cm length self-expanding nitinol stents were implanted into the same anatomic location in the contralateral iliofemoral artery (FIGS. 10E and 10F). A single, self-expanding, nitinol stent was deployed in a target porcine iliofemoral artery. Due to the inherent flexibility of the stent, the artery remained widely patent when the hind limb is extended (FIG. 10E) as well as maximally flexed (FIG. 10F). Numerical data from quantitative vascular angiographic analysis (QVA) are shown in each figure.

Following implantation, angiography was repeated with the hind limb in both extension and exaggerated flexion. Retrospective quantitative vascular analysis (QVA) was used to assess the morphology of the treated arteries. Measurements included treated artery lengths, diameters and bend angles during both hind limb extension and flexion. Axial compression was defined as the difference between arterial target segment length in the neutral, extended position minus the length in the flexed position divided by length in the neutral position. Bend angle was defined as the approximate angle between the proximal and distal border of the sample target arterial segment. The results showed that porcine iliofemoral arteries deformed markedly with hind limb flexion as expected. There was arterial extreme bending with hind limb flexion; no differences were noted in arteries treated with nitinol vs. the 5-scaffold device (FIG. 10G). Measurements were derived from angiographic images. Bend angle was defined as the approximate angle between the proximal and distal border of the sample target arterial segment. N=8 arteries. Data points represent mean±SEM.

Similarly, arteries deformed by manual flexion of the hind limb exhibited predictable axial compression. However, implantation of the 5-scaffold device allowed for more natural axial compression as opposed to longitudinally stiff nitinol devices (11% v. 1%; FIG. Measurements were derived from angiographic images. Axial compression was defined as the difference between arterial target segment lengths in the neutral, extended position minus the length in the flexed position divided by length in the neutral position. N=8 arteries. Data points represent mean±SEM.

Quantitative vascular angiographic diameter measurements were taken at 1 cm interval along the lengths of the treated arteries. As expected, the post-procedure lumen diameters were slightly greater after nitinol stenting due to the outward radial force generated by their self-expanding design (mean diameter 5.19±0.64 mm v. 4.38±0.55 mm). However, extreme flexion of the hind limb did not appreciably affect the diameter of either device (FIG. 10I). Measurements were derived from angiographic images. N=8 arteries. Data points represent mean±SEM. It was concluded from this experiment that this segmented, balloon-expandable stent systems effectively preserves the lumen during arterial bending.

Following implantation, animals in this study received oral acetylsalicylic acid 325 mg and clopidogrel 75 mg continuing daily. At each of the intervals of 30, 90, 180, 365, and 730 days, the animals were reanesthetized and the treated arteries reimaged. The results showed that arteries treated with control nitinol stents exhibited profound neointimal hyperplasia with luminal compromise and in-stent stenosis; in contrast, arteries treated with the 5-scaffold device exhibited only minimal stenosis and wide patency (FIGS. 10J and 10K). Angiographic images are shown of bilateral porcine femoral arteries treated with either a 5-scaffold, 60 mm device (FIG. 10J) or a 60 mm control nitinol stent (FIG. 10K) after 90-days. Note the wide patency of the EVSS compared to the critically stenotic metal stent (arrows). Notably, one distally-placed nitinol stent was found to be completely occluded at 90-days; in contrast, all of the 5-scaffold devices were widely patent at all time points of study.

Serial angiographic images were subjected to quantitative vascular analysis (QVA) to measure the development of arterial stenosis and lumen loss over time. Maximum percent diameter stenosis was calculated as (1−[MLD/RVD])×100%) where MLD=minimum lumen diameter and RVD=reference vessel diameter. The results showed that implantation of the 5-scaffold device in the porcine iliofemoral artery resulted in significant and sustained reductions in luminal stenosis (FIG. 10L). Angiographic Maximum Diameter Stenosis after implantation of the 5-scaffold device vs. nitinol SES in the porcine iliofemoral model are shown. N=8 arteries. Data points represent mean±SEM.

Serial optical coherence tomography (OCT) was utilized to image scaffold degradation over time. The scaffolds were fully covered after the first month, fully resorbed into the arterial wall after 6-mos. and fully degraded after 2-years (FIG. 10M).

After 2-years, the animals were sacrificed and the target arteries harvested for histologic and morphometric analysis. Arteries treated with the 5-scaffold device exhibited a moderate neointimal reaction (mean neointimal area 5.2±2.1 mm2) with preserved cytoarchitecture. The inter-scaffold spaces were largely free of vascular pathology. In contrast, arteries treated with nitinol SES exhibited significant neointimal reactions (mean neointimal area 12.7±5.2 mm2); in femoral arteries, nitinol struts could be observed extending beyond the external elastic lamina causing complete disruption of the arterial cytoarchitecture and flow-limiting stenosis (FIGS. 10N and 10O). Photomicrographs are shown of porcine femoral arteries treated with the 5-scaffold device (FIG. 10N) or nitinol stents (FIG. 10O) and harvested after 2 years. Note the mild neointimal response, preserved cytoarchitecture and lack of residual scaffolds in the artery treated with the 5-scaffold device (FIG. 10N). In contrast, note the gross disruption and luminal stenosis caused by the chronic outward force of the nitinol device (FIG. 10O). It was concluded from this study that the paclitaxel-eluting the 5-scaffold device significantly reduced neointima, late lumen loss and stenosis in a porcine model of percutaneous peripheral intervention.

Stents may be manufactured using an additive or a subtractive method. In any of the described embodiments, stents or stent elements may be manufactured as a sheet and wrapped into cylindrical form. Alternatively, stents or stent elements may be manufactured in cylindrical form using an additive manufacturing process. In an embodiment, stents maybe formed by extruding a material into a cylindrical tubing. In some embodiments, a longer stent element, may be formed during the manufacturing process and then cut into smaller stent elements/elements to provide a multi-element stent. In an embodiment, stent tubing may be laser cut with a pattern to form a stent element.

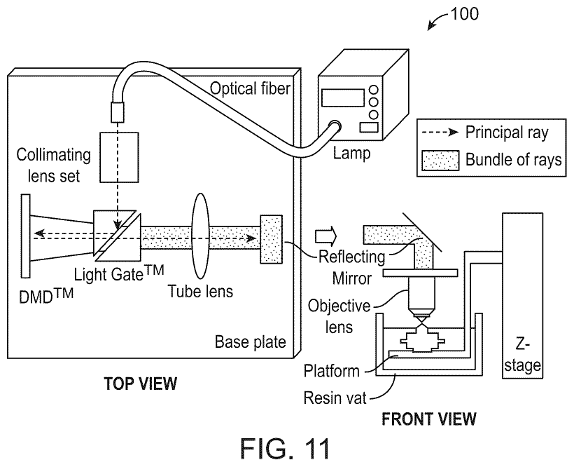

Referring now to FIG. 11, in one embodiment, stents may be manufactured using a micro-stereolithography system 100 (or “3D printing system”). Several examples of currently available systems that might be used in various embodiments include, but are not limited to: MakiBox A6, Makible Limited, Hong Kong; CubeX, 3D Systems, Inc., Circle Rock Hill, SC; and 3D-Bioplotter, (EnvisionTEC GmbH, Gladbeck, Germany).

The micro-stereolithography system may include an illuminator, a dynamic pattern generator, an image-former and a Z-stage. The illuminator may include a light source, a filter, an electric shutter, a collimating lens and a reflecting mirror that projects a uniformly intense light on a digital mirror device (DMD), which generates a dynamic mask. FIG. 10 shows some of these components of one embodiment of the micro-stereolithography system 100, including a DMD board, Z-stage, lamp, platform, resin vat and an objective lens. The details of 3D printing/micro-stereolithography systems and other additive manufacturing systems will not be described here, since they are well known in the art. However, according to various embodiments, any additive manufacturing system or process, whether currently known or hereafter developed, may potentially be used to fabricate stents within the scope of the present invention. In other words, the scope of the invention is not limited to any particular additive manufacturing system or process.

In one embodiment, the system 100 may be configured to fabricate stents using dynamic mask projection micro-stereolithography. In one embodiment, the fabrication method may include first producing 3D microstructural scaffolds by slicing a 3D model with a computer program and solidifying and stacking images layer by layer in the system. In one embodiment, the reflecting mirror of the system is used to project a uniformly intense light on the DMD, which generates a dynamic mask. The dynamic pattern generator creates an image of the sliced section of the fabrication model by producing a black-and-white region similar to the mask. Finally, to stack the images, a resolution Z-stage moves up and down to refresh the resin surface for the next curing. The Z-stage build subsystem, in one embodiment, has a resolution of about 100 nm and includes a platform for attaching a substrate, a vat for containing the polymer liquid solution, and a hot plate for controlling the temperature of the solution. The Z-stage makes a new solution surface with the desired layer thickness by moving downward deeply, moving upward to the predetermined position, and then waiting for a certain time for the solution to be evenly distributed.

Although particular embodiments have been shown and described, they are not intended to limit the invention. Various changes and modifications may be made to any of the embodiments, without departing from the spirit and scope of the invention. The invention is intended to cover alternatives, modifications, and equivalents.

What is claimed is:

1. A device for placement within a blood vessel to maintain or enhance blood flow through the blood vessel, the device comprising:
multiple balloon-expandable, bioresorbable, vascular stent elements configured to be implanted in the blood vessel as a stent;
wherein the stent elements are formed from a bioresorbable polymer material;
wherein the stent is configured to provide high radial force at the blood vessel wall while still preserving patency of the lumen during bending.

2. The device of claim 1, wherein bending of the blood vessel is accommodated by bending of spaces between the stent elements.

3. The device of claim 1, wherein axial compression of the blood vessel is absorbed by axial compression of both the stent elements and spaces between the stent elements.

4. The device of claim 1, wherein the bioresorbable polymer material comprises poly(L-lactic acid) (PLLA), poly(D-lactic acid) (PDLA), poly(D,L-lactic acid) (PDLLA), semi crystalline polylactide, polyglycolic acid (PGA), poly(lactic-co-glycolic acid) (PLGA), poly(iodinated desamino tyrosyl-tyrosine ethyl ester) carbonate, polycaprolactone (PCL), salicylate based polymer, polydioxanone (PDS), poly(hydroxybutyrate), poly(hydroxybutyrate-co-valerate), polyorthoester, polyanhydride, poly(glycolic acid-co-trimethylene carbonate), poly(iodinated desaminotyrosyl-tyrosine ethyl ester) carbonate, polyphosphoester, polyphosphoester urethane, poly(amino acids), cyanoacrylates, poly(trimethylene carbonate), poly(iminocarbonate), polyalkylene oxalates, polyphosphazenes, polyiminocarbonates, and aliphatic polycarbonates, fibrin, fibrinogen, cellulose, starch, collagen, polyurethane including polycarbonate urethanes, polyethylene, polyethylene terephthalate, ethylene vinyl acetate, ethylene vinyl alcohol, silicone including polysiloxanes and substituted polysiloxanes, polyethylene oxide, polybutylene terephthalate-co-PEG, PCL-co-PEG, PLA-co-PEG, PLLA-co-PCL, polyacrylates, polyvinyl pyrrolidone, polyacrylamide, or combinations thereof.

5. The device of claim 1, wherein the radial rigidity of the stent is slowly attenuated as its structural polymer is unlinked and metabolized such that the stent slowly becomes more flexible causing adaptation and remodeling of the vein and restoration of the vein's elasticity.
